(19) United States
(12) Patent Application Publication
(10) Pub. No.: US 2002/0086009 A1
(43) Pub. Date: Jul. 4, 2002

(54) ANTI-PHOSPHORYLATED TAU PROTEIN ANTIBODIES AND METHODS FOR DETECTING ALZHEIMER`S DISEASE WITH THE USE OF THE SAME

(76) Inventors: KOICHI ISHIGURO, TOKYO (JP); KAZUKI SATO, TOKYO (JP); JUN-MI PARK, TOKYO (JP); TSUNEKO UCHIDA, TOKYO (JP); KAZUTOMO IMAHORI, TOKYO (JP)

Correspondence Address: WENDEROTH LIND & PONACK, 2033 K STREET NW SUITE 800, WASHINGTON, DC, 20006

(21) Appl. No.: 09/142,613

(22) PCT Filed: Mar. 13, 1997
(86) PCT No.: PCT/JP97/00804

(30) Foreign Application Priority Data
Mar. 13, 1996 (JP) 8-56090

(51) Int. Cl. G01N 33/53; G01N 33/567; A61K 39/395

(52) U.S. Cl. 424/130.1; 435/7.2; 435/975; 424/141.1

(57) ABSTRACT

An antibody is prepared by using a partial peptide containing the phosphorylated site of a phosphorylated tau protein in the paired helical filament as an immunogen. Then the reactivity of the antibody thus obtained with samples obtained from individuals with a suspicion of Alzheimer's disease is examined to thereby detect the disease.

### TECHNICAL FIELD

The present invention relates to antibodies used for detecting Alzheimer's disease. More specifically, the present invention relates to antibodies to a partial peptide containing phosphorylation sites of phosphorylated tau protein in the paired helical filament, a reagent kit containing the same, and methods for detecting Alzheimer's disease using the antibodies or the kit.

### BACKGROUND ART

Alzheimer's disease is progressive dementia occurring at the presenile stage (the age of 45 to 65). It causes morbid changes such as degeneration of neurons and atrophia of cerebral cortex due to a decrease in the number of neurons. Pathologically, a number of senile plaques and neurofibrillary degeneration are observed in the brain. So-called senile dementia caused by spontaneous aging in the senium of the age of 65 or older is not substantially different from Alzheimer's disease from the pathological viewpoint and is regarded as senile dementia of Alzheimer type.

The number of patients suffering from Alzheimer's disease increases as senile population grows. This disease has thus drawn social attention. There are various hypotheses about the causes of this disease. However, it has not been elucidated yet and it is desired to be clarified soon.

The main component of senile plaques that is one of pathological changes caused by Alzheimer's disease is known to be amyloid β protein (Annu. Rev. Neurosci., 12, 463-490 (1989)). Neurofibrillary degeneration that is another pathological change shows accumulation of the paired helical filament in neurons and tau protein is identified as one of its constituents (J. Biochem. 99, 1807-1810 (1986); Proc. Natl. Acad. Sci. USA, 83, 4913-4917 (1986)).

Tau protein is composed of a group of protein isoforms that usually produce several bands at the molecular weight of 48 to 65 kD as a result of SDS-polyacrylamide gel electrophoresis and it is known to promote formation of microtubule. Tau protein incorporated in the PHF of the Alzheimer diseased brain was proved to be abnormally phosphorylated as compared with that in the normal brain using polyclonal antibody to PHF (anti-ptau; J. Biochem., 99, 1807-1810 (1986)) and monoclonal antibody to tau protein (tau-1 antibody;

Proc. Natl. Acad. Sci. USA, 83, 4913-4917 (1986)). The phosphorylation sites of phosphorylated tau protein incorporated in the PHF were also identified (JP 6-239893 A). Thus, functions of tau protein involved in Alzheimer's disease has being clarified.

However, it has not been known so far to detect Alzheimer's disease based on the phosphorylation site of phosphorylated tau protein in the PHF. Although methods for detecting Alzheimer's disease using various antibodies have been proposed, a clinically effective new detection method is still desired.

### DISCLOSURE OF THE INVENTION

The present inventors paid attention to the phosphorylation sites of phosphorylated tau protein in the PHF and found that antibodies obtained by using as an immunogen a partial peptide comprising a phosphorylated sites are useful for detecting Alzheimer's disease, thereby completing the present invention.

The present invention provides antibodies obtained by using as an immunogen a partial peptide comprising a phosphorylation sites of phosphorylated tau protein in the paired helical filament.

Preferred embodiments of this invention provides:

the antibody as described above, wherein the phosphorylation site of phosphorylated tau protein is one or more amino acid residues selected from serine at position 198, serine at position 199, serine at position 202, threonine at position 205, threonine at position 231, serine at position 235, serine at position 262, serine at position 396, threonine at position 403, serine at position 404, serine at position 409, serine at position 412, serine at position 413, and serine at position 422 of an amino acid sequence of SEQ ID NO: 1;

the antibody as described above, wherein the phosphorylation site of phosphorylated tau protein is one or more amino acid residues selected from serine at position 199, serine at position 202, threonine at position 205, threonine at position 231, serine at position 235, serine at position 262, serine at position 396, serine at position 404, serine at position 412, serine at position 413, and serine at position 422 of the amino acid sequence of SEQ ID NO: 1;

the antibody as described above, wherein the partial peptide comprising the phosphorylation site comprises an amino acid residue at the phosphorylated site and plural amino acid residues before and/or after sites of the phosphorylation site; and

the antibody as described above, wherein the partial peptide comprising the phosphorylation site has an amino acid sequence of any one of SEQ ID NO: 2 to SEQ ID NO: 16.

Another embodiment of this invention provides a reagent kit used for detecting Alzheimer's disease comprising at least any one of the above-described antibodies.

Yet another embodiment of this invention provides methods for detecting Alzheimer's disease comprises examining reactivity between any one of the above-described antibodies and a sample from an individual suspected of Alzheimer's disease.

The present invention will be described in detail below.

In the present invention, an example of tau protein is that having a primary structure consisting of 352 to 441 amino acid residues as described in Goedert et al., Neuron 3, 519-526 (1989). For example, when tau protein having the primary structure represented by the amino acid sequence shown in SEQ ID NO: 1 in the sequence listing is used, one or more amino acid residues to be phosphorylated are selected from serine at position 198, serine at position 199, serine at position 202, threonine at position 205, threonine at position 231, serine at position 235, serine at position 262, serine at position 396, threonine at position 403, serine at position 404, serine at position 409, serine at position 412, serine at position 413, and serine at position 422 of the same sequence (J. Biol. Chem., 270, 823-829 (1995) and Neurosci. Lett., 189, 167-170 (1995)).

It is preferable to use a partial peptide of tau protein comprising one or more phosphorylated amino acid residues selected from serine at position 199, serine at position 202, threonine at position 205, threonine at position 231, serine at position 235, serine at position 262, serine at position 396, serine at position 404, serine at position 412, serine at position 413 and serine at position 422 of the above-described sequence.

A peptide comprising the amino acid residue(s) at the above-described phosphorylated site(s) and plural amino acid residues before and/or after sites of the phosphorylation site(s) is preferably used as the partial peptide of tau protein used in the present invention. In particular, such a peptide comprises preferably 1 to 7 amino acid residues, more preferably 3 to 5 amino acid residues, at either before or after site or both sites of the amino acid residue at the phosphorylation site. Among these partial peptides, the peptide having the amino acid sequence described in any one of SEQ ID NO: 2 to 16 in the sequence listing is most preferably used.

The antibody of the present invention can be obtained by immunizing an animal with the partial peptide comprising the phosphorylation site of the phosphorylated tau protein of the present invention as an immunogen and preparing serum from the animal. In this occasion, it is preferable to use as an immunogen a carrier protein to which the above-described peptide having introduced at its amino terminus or carboxyl terminus an amino acid residue with a reactive functional group, such as cysteine, lysine, glutamic acid, or aspartic acid, is bound as a hapten.

Among the above-described peptides, the partial peptides represented by SEQ ID NO: 3, 13, 15, and 16 are synthesized by the solid phase peptide synthesis using a phenyl group as a protective group for the phosphorylation site as described in Tetrahedron Lett., 32, 7083-7086 (1991).

The phosphorylated peptides represented by the other sequence than those represented by the above-described sequence identification number, which have an aromatic amino acid, a sulfur-containing amino acid, or a heterocyclic amino acid, are synthesized by the solid phase peptide synthesis using a cyclohexyl group as a protective group for the phosphorylation site as described in Peptide Chemistry 1993, 109-112 (1994) or by the solid phase peptide synthesis using a benzyl group as a protective group for the phosphorylation site as described in Chem. Lett., 1099-1112 (1994).

In the preparation of the antibody, the partial peptide synthesized as described above is bound to a carrier protein such as bovine serum albumin (BSA), thyroglobulin, or keyhole lympet hemocyanin. The binding can be performed easily using an appropriate condensing agent, such as maleimide, glutaraldehyde, or carbodiimide. The thus-obtained peptide bound to a carrier protein is used to immunize an animal. The immunization of an animal can be performed in the same method as conventional methods used for antibody production. More specifically, a solution comprising the peptide bound to a carrier protein is mixed with adjuvant if necessary, and subcutaneously or intraperitoneally administered to animals usually used for antibody production, such as mice, rats, rabbits, guinea pigs, or sheep. Additional immunization is performed every two to three weeks after the first immunization to produce antiserum with a high antibody titer. Blood is collected from the immunized animal to prepare serum. In the present invention, the thus-obtained antiserum can be used as it is without purification. Alternatively, serum may be heat-treated to inactivate complements and subjected to salting out with ammonium sulfate and ion exchange chromatography to purify immunoglobulin-containing fractions. Furthermore, the antibody may be purified using a peptide column having a specific partial peptide immobilized to obtain an antibody specifically recognizing the above-described phosphorylation site or its vicinity.

A monoclonal antibody recognizing a specific epitope can be obtained by collecting antibody-producing cells from the immunized animal, subjecting the collected cells to cell fusion with cultured cells such as a myeloma cell line derived from the same animal to prepare hybridoma, and preparing immunoglobulin fractions from the culture medium of the hybridoma by conventional methods.

Alzheimer's disease can be detected by immunologically reacting the antibody obtained by the present invention with a sample derived from an individual suspected of Alzheimer's disease by per se known conventional methods, detecting an antigen-antibody reaction product, and examining reactivity between the sample and the antibody. The antibody obtained as described above can be used to prepare a reagent kit for detecting Alzheimer's disease by the above-described method comprising the immunoreaction and the detection step. Such a kit comprises the constituents usually used in the immunoreaction-based kit. More specifically, the reagent kit of the present invention comprises at least the antibody of the present invention and, as optional ingredients, a solution for diluting a sample, a washing solution, a labeled antibody or a labeled antigen, chromogen, and a peptide for positive control.

Alzheimer's disease can be detected using the antibody or the reagent kit of the present invention for example as follows.

A sample is first obtained from an individual suspected of Alzheimer's disease and reacted with the antibody obtained as described above. The sample may be tissues from cerebral cortex or the like and body fluid such as cerebrospinal fluid or blood. When a tissue sample is subjected to the detection method of the present invention, about 0.1 mg of the sample is required. When cerebrospinal fluid or blood is used as a sample, about 0.5 to 0.01 ml of the sample is required.

Once the above-described sample is obtained, the sample is homogenized in physiological saline and centrifuged. The resulting supernatant is fractionated to remove contaminating immunoglobulin and examined for reactivity to the above-obtained antibody as an index.

The thus-obtained fraction is electrophoresed. The antibody as obtained above is added thereto to perform immunoblotting. In this occasion, the antibody can be detected by labeling it with the label used conventionally. Alternatively, the antibody may be detected by reacting it with a secondary antibody that is reactive with the antibody.

If a sample from an individual suspected of Alzheimer's disease is examined for reactivity with the antibody and the reactivity increases compared with a control derived from an individual without Alzheimer's disease, the individual is regarded to be a patient of Alzheimer's disease. If the reactivity decreases compared with a control from an individual with Alzheimer's disease, the individual is regarded to be free from Alzheimer disease. In this way, Alzheimer's disease can be detected by the present invention.

In the present invention, unlike an antibody obtained using as an immunogen phosphorylated tau protein as a whole or the PHF, the antibody highly specific to each phosphorylation site can be obtained. This antibody is useful for detection specific to the phosphorylation site of phosphorylated tau protein without determining antigen-recognizing specificity. Furthermore, various antibodies site-specific to each phosphorylation site can be efficiently obtained. The thus-obtained various antibodies are examined for reactivity with a sample from an individual with Alzheimer's disease to select an antibody most suitable for detection of Alzheimer's disease easily.

### BRIEF DESCRIPTION OF THE DRAWINGS

FIG. 1 is the dot blot showing specificity of the antibodies obtained by immunization with a partial peptide containing a phosphorylation site of phosphorylated tau protein.

FIG. 2 is photographs of electrophoresis (immunoblotting) showing reactivity of the TS fraction (the fraction obtained by removing IgG from the supernatant of human cerebral cortex suspension) obtained in Example with the antibodies used in the present invention.

FIG. 3 is photographs of electrophoresis (immunoblotting) showing reactivity of the SDS precipitation fraction obtained in Example with the antibodies used in the present invention.

FIG. 4 is photographs of electrophoresis (immunoblotting) showing reactivity of the SDS precipitation fraction obtained in Example with the antibodies used in the present invention.

FIG. 5 shows a calibration curve in the competitive RIA obtained in Example.

FIG. 6 shows the results of measuring the concentrations of phosphorylated tau protein in the cerebrospinal fluid from patients with Alzheimer's disease and patients with no dementia obtained in Example.

### BEST MODE FOR CARRYING OUT THE INVENTION

The present invention will be described below in more detail with reference to Examples, but is not construed to be limited thereto.

### PRODUCTION EXAMPLE 1

Preparation of Partial Peptide Having Amino Acid Sequence Described in SEQ ID NO: 3 in the Sequence Listing (This Peptide and the Antibodies Against this Peptide are Hereinafter Sometimes Referred to as “PS202” and “Anti-PS202”, Respectively.)

A peptide shown by H-Lys-Ser-Ser-Pro-Gly-Ser(H₂PO₃)-Pro-Gly-Thr-Pro-Gly-Ser-Arg-NH₂ (SEQ ID NO: 3) was produced by the following method. Symbols hereinafter used respectively have the following meanings: MBHA resin: p-methylbenzhydrylamine resin; Boc: t-butyloxycarbonyl group; Bzl: benzyl group; Ph: phenyl group; Tos: p-toluenesulfonyl group; and Z(2-Cl): 2-chlorobenzyloxycarbonyl group.

(a) Production of H-Lys[Z(2-Cl)]-Ser(Bzl)-Ser(Bzl)-Pro-Gly-Ser[PO(OPh)₂]-Pro-Gly-Thr(BzL)-Pro-Gly-Ser(Bzl)-Arg(T os)-MBHA Resin

0.94 g of MBHA resin (amine content: 0.64 mmol/g resin) was set in the Bio Search 9500 Model Automatic Peptide Synthesizer. Boc-Arg(Tos)-OH, Boc-Ser(Bzl)-OH, Boc-Gly-OH, Boc-Pro-OH, Boc-Thr(Bzl)-OH, Boc-Gly-OH, Boc-Pro-OH, Boc-Ser[PO(OPh)₂]-OH, Boc-Gly-OH, Boc-Pro-OH, Boc-Ser(Bzl)-OH, Boc-Ser(Bzl)-OH, and Boc-Lys[Z(2-Cl)]-OH were supplied thereto and were then coupled in this order using diisopropylcarbodiimide as a condensing agent to obtain 2.38 g of the above-described side chain-protected peptide-MBHA resin.

(b) Treatment with Hydrogen Fluoride

1.34 g of the side chain-protected peptide-MBHA resin obtained in (a) was collected and set in a hydrogen fluoride reaction device manufactured by the Peptide Institute, Inc. The resin was reacted with 13 ml of hydrogen fluoride in the presence of 1.5 ml anisole under ice-cooling for 1 hour. After the completion of the reaction, hydrogen fluoride was distilled off under reduced pressure. The residue was washed with ethyl acetate and extracted with 150 ml of 2 M acetic acid to obtain 350 mg of a crude peptide having a protected phosphate group and shown by H-Lys-Ser-Ser-Pro-Gly-Ser[PO(OPh)₂]-Pro-Gly-Thr-Pro-Gly-ser-Arg-NH₂ .

This product was dissolved in 20 ml of 30% acetic acid and the resulting solution was applied to a Sephadex G-25 column (internal diameter: 5 cm and length: 109 cm). Elution was performed using the same solvent to collect the fraction containing the desired product. The thus-obtained fraction was then dissolved in a small amount of distilled water and purified by HPLC using a reverse phase column (internal diameter: 2 cm and length: 25 cm) packed with ODS (octadecylsilane)-bound silica. Elution was performed by a linear gradient of 5 to 65% acetonitrile in 0.1% trifluoroacetic acid. The yield of the purified product was 110 mg. The structure of this substance was confirmed by FAB mass spectrometry; measured [M+H]⁺; 1445, calculated (C₆₁H₉₂N₁₈O₂₁P₁+H); 1445.

(c) Hydrogenolysis

90 mg of the phosphate group-protected peptide obtained in (b) and 80 mg of platinum oxide (catalyst) were mixed with 1 ml of acetic acid and the mixture was stirred at room temperature for 12 hours under hydrogen atmosphere of 5 to 6 pressure. After the catalyst was filtered off, the filtrate and washings were collected and lyophilized. The resulting product was purified by preparatory HPLC to obtain 55 mg of a final product, phosphorylated peptide shown by H-Lys-Ser-Ser-Pro-Gly-Ser[H₂PO₃]-Pro-Gly-Thr-Pro-Gly-Ser-Arg-NH₂. The structure of this substance was confirmed by FAB mass spectrometry; measured [M+H]⁺; 1294, calculated (C₄₉H₈₅N₁₈O₂₁P₁+H); 1294.

### PRODUCTION EXAMPLES 2 TO 4

Partial peptides having amino acid sequences described in SEQ ID NO: 13, NO: 15, and NO: 16 in the sequence listing were obtained in the same manner as described in Production Example 1 (These peptides are hereinafter sometimes referred to as “PS413”, “PS412”, and “PS412, 413” and the antibodies to these peptides are referred to as “anti-PS413”, “anti-PS412”, and “anti-PS412, 413”, respectively.)

### PRODUCTION EXAMPLE 5

Preparation of Partial Peptide Having Amino Acid Sequence Described in SEQ ID NO: 2 in the Sequence Listing (This Peptide and the Antibodies to this Peptide are Hereinafter Sometimes Referred to as “PS199” and “Anti-PS199”, Respectively.)

A peptide shown by H-Lys-Ser-Gly-Tyr-Ser-Ser(H₂PO₃)-Pro-Gly-Ser-Pro-Gly-Thr-NH₂ (SEQ ID NO: 2) was produced by the following method. Symbols used hereinafter respectively have the following meanings: MBHA resin: p-methylbenzhydrylamine resin; Boc: t-butyloxycarbonyl group; Bzl: benzyl group; cHex: cyclohexane group; Z(2-Br): 2-bromobenzyloxycarbonyl group; and Z(2-Cl): 2-chlorobenzyloxycarbonyl group.

(a) Production of H-Lys[Z(2-Cl)]-Ser(Bzl)-Gly-Tyr[Z(2-Br )]-Ser(Bzl)-Ser[PO(OcHex)₂]-Pro-Gly-Ser(BzL)-Pro-Gly-Thr (Bzl)-MBHA Resin

131 mg of MBHA resin (amine content: 0.76 mmol/g resin) was set in the Bio Search 9500 Model Automatic Peptide Synthesizer. Boc-Thr(Bzl)-OH, Boc-Gly-OH, Boc-Pro-OH, Boc-Ser(Bzl)-OH, Boc-Gly-OH, Boc-Pro-OH, Boc-Ser[PO(OcHex)₂]-OH, Boc-Ser(Bzl)-OH, Tyr[Z(2-Br)]-OH, Boc-Gly-OH, Boc-Ser(Bzl)-OH, and Boc-Lys[Z(2-Cl)]-OH were supplied thereto and were then coupled in this order using diisopropylcarbodiimide as a condensing agent to obtain 307 mg of the above-described side chain-protected peptide-MBHA resin.

(b) Treatment with Trifluoromethanesulfonic Acid

150 mg of the side chain-protected peptide-MBHA resin obtained in (a) was collected. 10 ml of trifluoroacetic acid containing 1 M methanesulfonic acid and thioanisole, and 0.05 ml of m-cresol were added thereto and the resulting mixture was allowed to react for 4 hours under ice-cooling. After the completion of the reaction, 200 ml of ice-cooled diethyl ether was added thereto to precipitate a peptide. The whole content was collected by filtration with a glass filter and washed with cold diethyl ether. The residue was extracted with 200 ml of 2 M acetic acid to obtain 53 mg of a crude peptide shown by H-Lys-Ser-Gly-Tyr-Ser-Ser[H₂PO₃]-Pro-Gly-Ser-Pro-Gly-Thr-NH₂.

(c) Purification of the Peptide

This product was dissolved in 6 ml of distilled water and purified by HPLC using a reverse phase column (internal diameter: 2 cm and length: 25 cm) packed with ODS (octadecylsilane)-bound silica. Elution was performed by a linear gradient of 5 to 35% acetonitrile in 0.1% trifluoroacetic acid. The yield of the purified product was 29 mg. The structure of this substance was confirmed by FAB mass spectrometry; measured [M+H]⁺; 1204, calculated (C₄₇H₇₅N₁₄O₂₁P₁+H); 1204.

### PRODUCTION EXAMPLE 6

Preparation of Partial Peptide Having Amino Acid Sequence Described in SEQ ID NO: 6 in the Sequence Listing (This Peptide and the Antibodies to this Peptide are Hereinafter Sometimes Referred to as “PT231” and “Anti-PT231”, Respectively.)

A peptide shown by H-Cys-Val-Ala-Val-Val-Arg-Thr(H₂PO₃)-Pro-Pro-Lys-Ser-Pro-Ser-Ser-OH (SEQ ID NO: 6) was produced by the following method. Symbols used hereinafter respectively have the following meanings: Bzl resin: benzyl alcohol resin; Boc: t-butyloxycarbonyl group; Bzl: benzyl group; MBzl: 4-methoxybenzyl group; Mts: methylenesulfonyl group; cHex: cyclohexyl group; and Z(2-Cl): 2-chlorobenzyloxycarbonyl group.

(a) Production of H-Cys(MBzl)-Val-Ala-Val-Val-Arg(Mts)-Thr[PO(OcHex)₂]-Pro-Pro-Lys[Z(2-Cl)]-Ser(BzL)-Pro-Ser(Bzl)-Ser(Bzl)-Bzl Resin

71 mg of Boc-Ser(Bzl)-Bzl resin (amine content: 0.70 mmol/g resin) was set in the Bio Search 9500 Model Automatic Peptide Synthesizer. Boc-Ser(Bzl)-OH, Boc-Pro-OH, Boc-Ser(Bzl)-OH, Boc-Lys[Z(2-Cl)]-OH, Boc-Pro-OH, Boc-Pro-OH, Boc-Thr[PO(OcHex)₂]-OH, Boc-Arg(Mts)-OH, Boc-Val-OH, Boc-Val-OH, Boc-Ala-OH, Boc-Val-OH, and Boc-Cys(MBzl)-OH were supplied thereto and were then coupled in this order using diisopropylcarbodiimide as a condensing agent to obtain 62 mg of the above-described side chain-protected peptide-Bzl resin.

(b) Treatment with Trifluoromethanesulfonic Acid

To 62 mg of the side chain-protected peptide-Bzl resin obtained in (a) were added 0.9 ml of trifluoromethanesulfonic acid, 1.2 ml of thioanisole, 6.6 ml of trifluoroacetic acid, 0.9 ml of m-cresol, and 0.4 ml of ethanedithiol. The mixture was allowed to react for 5 minutes under ice-cooling and then at room temperature for 3 hours. After the completion of the reaction, 200 ml of ice-cooled diethyl ether was added thereto to precipitate a peptide. The whole content was collected by filtration with a glass filter and washed with cold diethyl ether. The residue was extracted with 170 ml of 2 M acetic acid to obtain 21 mg of a crude peptide shown by H-Cys-Val-Ala-Val-Val-Arg-Thr(H₂PO₃)-Pro-Pro-Lys-Ser-Pro-Ser-Ser-OH.

(c) Purification of the Peptide

This product was dissolved in 10 ml of 30% acetic acid and applied to a Sephadex G-25 column (internal diameter: 5 cm and length: 107 cm). Elution was performed using the same solvent to collect the fractions containing the desired product. The yield of this purified product was 12 mg.

The thus-obtained product was dissolved in 5 ml of 30% acetic acid and purified by HPLC using a reverse phase column (internal diameter: 2 cm and length: 25 cm) packed with ODS (octadecylsilane)-bound silica. Elution was performed with 13% acetonitrile in 0.1% trifluoroacetic acid. The yield of the purified product was 7 mg. The structure of this substance was confirmed by FAB mass spectrometry; measured [M+H]⁺; 1509, calculated (C₆₁H₁₀₇N₁₈O₂₂P₁S₁+H): 1508.

### PRODUCTION EXAMPLE 7

Preparation of Partial Peptide Having Amino Acid Sequence Described in SEQ ID NO: 11 in the Sequence Listing (This Peptide and the Antibodies to this Peptide are Hereinafter Sometimes Referred to as “PS396” and “Anti-PS396”, Respectively.)

A peptide shown by H-Cys-Glu-Ile-Val-Tyr-Lys-Ser(H₂PO₃)-Pro-Val-Val-Ser-Gly-NH₂ (SEQ ID NO: 11) was produced by the following method. Symbols used hereinafter respectively have the following meanings: MBHA resin: p-methylbenzhydrylamine resin; Boc: t-butyloxycarbonyl group; Bzl: benzyl group; MBzl, 4-methoxybenzyl group; cHex: cyclohexyl group; Z(2-Br): 2-bromobenzyloxycarbonyl group; and Z(2-Cl): 2-chlorobenzyloxycarbonyl group.

(a) Production of H-Cys(MBzl)-Glu(OBzl)-Ile-Val-Tyr[Z(2-Br)]-Lys[Z(2-Cl)]-Ser[PO(OcHex) 2 ]-Pro-Val-Val-Ser(Bzl)-Gly-MBHA Resin

131 mg of MBHA resin (amine content: 0.76 mmol/g resin) was set in the Bio Search 9500 Model Automatic Peptide Synthesizer. Boc-Gly-OH, Boc-Ser(Bzl)-OH, Boc-Val-OH, Boc-Val-OH, Boc-Pro-OH, Boc-Ser[PO(OcHex)₂ ]-OH, Boc-Lys[Z(2-Cl)]-OH, Tyr[(Z(2-Br)]-OH, Boc-Val-OH, Boc-Ile-OH, Boc-Glu(OBzl)-OH, and Boc-Cys(MBzl)-OH were supplied thereto and were then coupled in this order using diisopropylcarbodiimide as a condensing agent to obtain 376 mg of the above-described side chain-protected peptide-MBHA resin.

(b) Treatment with Trifluoromethanesulfonic Acid

To 188 mg of the side chain-protected peptide-MBHA resin obtained in (a) were added 10 ml of trifluoroacetic acid containing 1 M methanesulfonic acid and thioanisole, and 0.05 ml of m-cresol. The mixture was allowed to react for 4 hours under ice-cooling. After the completion of the reaction, 200 ml of ice-cooled diethyl ether was added thereto to precipitate a peptide. The whole content was collected by filtration with a glass filter and washed with cold diethyl ether, and extracted with 200 ml of 2 M acetic acid to obtain 87 mg of a crude peptide shown by H-Cys-Glu-Ile-Val-Tyr-Lys-Ser(H₂PO₃)-Pro-Val-Val-Ser-Gly-NH_{2.}

(c) Purification of the Peptide

This product was dissolved in 9 ml of 30% acetic acid and purified by HPLC using a reverse phase column (internal diameter: 2 cm and length: 25 cm) packed with ODS (octadecylsilane)-bound silica. Elution was performed with 16% acetonitrile in 0.1% trifluoroacetic acid. The yield of the purified product was 45 mg. The structure of this substance was confirmed by FAB mass spectrometry; measured [M+H]⁺; 1360, calculated (C₅₇H₉₅N₁₄O₂₀P₁S₁+H); 1360.

### PRODUCTION EXAMPLES 8 TO 10

Partial peptides having amino acid sequence described in SEQ ID NO: 4, NO: 12, and NO: 14 in the sequence listing were obtained in the same manner as in Production Examples 5, 6, and 7 (These peptides are hereinafter referred to as “PT205”, “PS404”, and “PS422”, respectively, and antibodies to these peptides are referred to as “anti-PT205”, “anti-PS404”, and “anti-PS422”, respectively.)

### PRODUCTION EXAMPLE 11

Preparation of Partial Peptide Having Amino Acid Sequence Described in SEQ ID NO: 7 in the Sequence Listing (This Peptide and the Antibodies to this Peptide are Hereinafter Sometimes Referred to as “PS235” and “Anti-PS235”, Respectively.)

A peptide shown by H-Cys-Arg-Thr-Pro-Pro-Lys-Ser(H₂PO₃)-Pro-Ser-Ser-Ala-Lys-OH (SEQ ID NO: 7) was produced by the following method. Symbols used hereinafter respectively have the following meanings: Alko resin: p-alkoxybenzyl alcohol resin; Boc: t-butyloxycarbonyl group; tBu: t-butyl group; Bzl: benzyl group; Fmoc: 9-fluorenylmethoxycarbonyl group; Trt: trityl group; Pmc: pentamethylchroman-6-sulfonyl group.

(a) Production of H-Cys(Trt)-Arg(Pmc)-Thr(tBu)-Pro-Pro-Lys(Boc)-Ser[PO(OH)(OBzl)]-Pro-Ser(tBu)-Ser(tBu)-Ala-Lys(Boc)-Alko Resin

385 mg of Fmoc-Lys(Boc)-Alko resin (amino acid content: 0.65 mmol/g resin) was set in the Applied Bio Systems 431 Model Automatic Peptide Synthesizer. Fmoc-Ala-OH, Fmoc-Ser(tBu)-OH, Fmoc-Ser(tBu)-OH, Fmoc-Pro-OH, Fmoc-Ser[PO(OH)(OBzl)]-OH, Fmoc-Lys(Boc)-OH, Fmoc-Pro-OH, Fmoc-Pro-OH, Fmoc-Thr(tBu)-OH, and Fmoc-Arg(Pmc)-OH were supplied thereto and were then coupled in this order using HBTU [2-(1H-benzotriazole-1-yl)-1,1,3,3-tetr amethyluronium hexafluorophosphate] as a condensing agent to obtain 716 mg of a side chain-protected peptide-Alko resin intermediate. Fmoc-Cys(Trt)-OH was condensed with 358 mg of this intermediate to obtain 395 mg of the above-described side chain-protected peptide-Alko resin.

(b) Treatment With Trifluoacetic Acid

To 196 mg of the side chain-protected peptide-Alko resin obtained in (a) was added a mixture containing 8.25 ml of trifluoroacetic acid, 0.5 ml of purified water, 0.5 ml of thioanisole, 0.75 ml of phenol, and 0.25 ml of ethanedithiol. The resulting mixture was allowed to react for 1.5 hour at room temperature. After the completion of the reaction, 200 ml of ice-cooled diethyl ether was added thereto to precipitate a peptide. The whole content was collected by filtration with a glass filter, washed with cold diethyl ether, and extracted with 80 ml of 2 M acetic acid to obtain 82 mg of a crude peptide shown by H-Cys-Arg-Thr-Pro-Pro-Lys-Ser(H₂PO₃)-Pro-Ser-Ser-Ala-Lys-OH.

(c) Purification of the Peptide

This product was dissolved in 7 ml of 0.1% trifluoroacetic acid and purified by HPLC using a reverse phase column (internal diameter: 2 cm and length: 25 cm) packed with ODS (octadecylsilane)-bound silica. Elution was performed with 7% acetonitrile in 0.1% trifluoroacetic acid. The yield of the purified product was 62 mg. The structure of this substance was confirmed by FAB mass spectrometry; measured [M+H]⁺; 1339, calculated (C₅₂H₉₂N₁₇O₂₀P₁S₁+H); 1339.

### PRODUCTION EXAMPLES 12 to 15

Partial peptides having amino acid sequence described in SEQ ID NO: 5, NO: 8, NO: 9, and NO: 10 in the sequence listing were obtained in the same manner as in Production Example 11 (These peptides are hereinafter sometimes referred to as “PS199,202”, “ratPS235”, “PT231,PS235”, and “PS262”, respectively, and the antibodies to these peptides are referred to as “anti-PS199,202”, “anti-ratPS235”, “anti-PT231,PS235”, and “anti-PS262”, respectively.)

### PRODUCTION EXAMPLE 16

Preparation of Partial Peptide Having Amino Acid Sequence Described in SEQ ID NO: 17 in the Sequence Listing (This Peptide and the Antibodies to this Peptide are Hereinafter Sometimes Referred to as “Tau-C” and “Anti-Tau-C”, Respectively.)

A peptide shown by H-Ser-Pro-Gln-Leu-Ala-Thr-Leu-Ala-Asp-Glu-Val-Ser-Ala-Ser-Leu-Ala-Lys-OH (SEQ ID NO: 17) was produced by the following method. Symbols used hereinafter respectively have the following meanings: Alko resin: p-alkoxybenzyl alcohol resin; Boc: t-butyloxycarbonyl group; tBu: t-butyl group; Bzl: benzyl group; Fmoc: 9-fluorenylmethoxycarbonyl group; and Trt: trityl group.

(a) Production of H-Ser(tBu)-Pro-Gln(Trt)-Leu-Ala-Thr(tBu)-Leu-Ala-Asp(OtBu)-Glu(OtBu)-Val-Ser(tBu)-Ala-Ser-Leu-Ala-Lys(Boc)-Alko Resin

284 mg of Alko resin (amine content: 0.88 mmol/g resin) was set in the ABI A431 Model Automatic Peptide Synthesizer. Fmoc-Lys(Boc)-OH was bound to the resin using dimethylaminopyridine and diisopropylcarbodiimide as condensing agents. Then, Fmoc-Ala-OH, Fmoc-Leu-OH, Fmoc-Ser(tBu)-OH, Fmoc-Ala-OH, Fmoc-Ser(tBu)-OH, Fmoc-Val-OH, Fmoc-Glu(OtBu)-OH, Fmoc-Asp(OtBu)-OH, Fmoc-Ala-OH, Fmoc-Leu-OH, Fmoc-Thr(tBu)-OH, Fmoc-Ala-OH, Fmoc-Leu-OH, Fmoc-Gln(Trt)-OH, Fmoc-Pro-OH, and Fmoc-Ser(tBu)-OH were supplied thereto and were then coupled in this order using HBTU [2-(1H-benzotriazole-1-yl)-1,1,3,3-tetramethyluronium hexafluorophosphate] as a condensing agent to obtain 905 mg of the above-described side chain-protected peptide-Alko resin.

(b) Treatment with Trifluoroacetic Acid

To 543 mg of the side chain-protected peptide-Alko resin obtained in (a) were added 9.5 ml of trifluoroacetic acid, 0.25 ml of ethanedithiol, and 0.5 ml of distilled water. The mixture was allowed to react for 5 minutes under ice-cooling and then at room temperature for 1.5 hour. After the completion of the reaction, 200 ml of ice-cooled diethyl ether was added thereto to precipitate a peptide. The whole content was collected by filtration with a glass filter, washed with cold diethyl ether, and extracted with 100 ml of 2 M acetic acid to obtain 250 mg of a crude peptide shown by H-Ser-Pro-Gln-Leu-Ala-Thr-Leu-Ala-Asp-Glu-Val-Ser-Ala-Ser-Leu-Ala-Lys-OH.

(c) Purification of the Peptide

This product was dissolved in 20 ml of 30% acetic acid and applied to a Sephadex G-25 column (internal diameter: 5 cm and length: 107 cm). Elution was performed using the same solvent to collect the fractions containing the desired product. The yield of this purified product was 122 mg. The structure of this substance was confirmed by FAB mass spectrometry; measured [M+H]⁺; 1702, calculated (C₇₃H₁₂₅N₁₉O₂₇S₂+H); 1701.

### PRODUCTION EXAMPLE 17

Preparation of Partial Peptide Having Amino Acid Sequence Described in SEQ ID NO: 18 in the Sequence Listing (This Peptide and the Antibodies to this Peptide are Hereinafter Sometimes Referred to as “Tau-N” and “Anti-Tau-N”, Respectively.)

A peptide shown by H-Ala-Glu-Pro-Arg-Gln-Glu-Glu-Phe-Glu-Val-Met-Glu-Cys-NH₂ (SEQ ID NO: 18) was produced by the following method. Symbols used hereinafter respectively have the following meanings: Fmoc-NH-SAL resin: 4-(2′,4′-dimethoxyphenyl-Fmoc-aminoethyl)phenoxy resin; Boc: t-butyloxy-carbonyl group; tBu: t-butyl group; Bzl: benzyl group; Fmoc: 9-fluorenylmethoxycarbonyl group; Trt: trityl group; and Pmc: pentamethylchroman-6-sulfonyl group.

(a) Production of H-Ala-Glu(OtBu)-Pro-Arg(Pmc)-Gln(Trt)-Glu(OtBu)-Phe-Glu(OtBu)-Val-Met-Glu(OtBu)-Cys(Trt)-NH-SAL Resin

532 mg of Fmoc-NH-SAL resin (amine content: 0.47 mmol/g resin) was set in the ABI A431 Model Automatic Peptide Synthesizer. Fmoc-Cys(Trt)-OH, Fmoc-Glu(OtBu)-OH, Fmoc-Met-OH, Fmoc-Val-OH, Fmoc-Glu(OtBu)-OH, Fmoc-Phe-OH, Fmoc-Glu(OtBu)-OH, Fmoc-Gln(Trt)-OH, Fmoc-Arg(Pmc)-OH, Fmoc-Pro-OH, Fmoc-Glu(OtBu)-OH, and Fmoc-Ala-OH were supplied thereto and were then coupled in this order using HBTU [2-(1H-benzotriazole-1-yl)-1,1,3, 3-tetramethyluronium hexafluorophosphate] as a condensing agent to obtain 1122 mg of the above-described side chain-protected peptide-NH-SAL resin.

(b) Treatment with Trifluoroacetic Acid

To 673 mg of the side chain-protected peptide-NH-SAL resin obtained in (a) were added 0.75 ml of phenol, 0.5 ml of thioanisole, 8.25 ml of trifluoroacetic acid, 0.25 ml of ethanedithiol, and 0.5 ml of distilled water. The mixture was allowed to react for 5 minutes under ice-cooling and then at room temperature for 1.5 hour. After the completion of the reaction, 200 ml of ice-cooled diethyl ether was added thereto to precipitate a peptide. The whole content was collected by filtration with a glass filter, washed with cold diethyl ether, and extracted with 50 ml of 2 M acetic acid and 250 ml of distilled water to obtain 182 mg of a crude peptide shown by H-Ala-Glu-Pro-Arg-Gln-Glu-Glu-Phe-Glu-Val-Met-Glu-Cys-NH₂.

(c) Purification of the Peptide

This product was dissolved in 20 ml of 30% acetic acid and applied to a Sephadex G-25 column (internal diameter: 5 cm and length: 107 cm). Elution was performed using the same solvent to collect the fractions containing the desired product. The yield of this purified product was 136 mg.

This product was dissolved in 20 ml of 20% acetonitrile and purified by HPLC using a reverse phase column (internal diameter: 2 cm and length: 25 cm) packed with ODS (octadecylsilane)-bound silica. Elution was performed with 22% acetonitrile in 0.1% trifluoroacetic acid. The yield of the purified product was 96 mg. The structure of this substance was confirmed by FAB mass spectrometry; measured [M+H]⁺; 1467, calculated (C₆₁H₉₅N₁₇O₂₁S₂+H); 1467.

### EXAMPLE 1

### Preparation of Antibodies

The partial peptides obtained in Production Examples 1 to 17 were each bound to an equivalent weight of keyhole lympet hemocyanin to serve as an immunogen. 0.2 mg of each of the immunogens was dissolved in 0.3 ml of physiological saline and emulsified with an equivalent volume of Freund's adjuvant. Rabbits were immunized with the resulting emulsion every three weeks. The thus-obtained antisera were purified by applying onto a Affigel 15 column (Bio-Rad) to which the respective antigenic peptide was bound and eluted using Immunopure gentle Ag/Ab buffer system (Pierce). Thus, the above-described antibodies that specifically recognize the respective phosphorylation sites were obtained.

Specificity of the antibodies was confirmed by dot blotting.

More specifically, Immobilon P-membrane (Millipore) was dotted with 18 pmol of each peptide dissolved in a 70% DMSO solution in line with each other, allowed to adsorb them, and dried. This membrane was immersed in TBS (20 mM Tris-HCl (pH 7.5), 150 mM NaCl) containing 5% skim milk for 1 hour to prevent non-specifically binding of the antibodies to be added in the following step to the membrane. The membrane was then washed with TBS for 5 minutes three times, immersed in TBS containing the desired antibody (first antibody), placed between parafilm, and allowed to react at 4° C. for 14 hours in a humidified box to bind the first antibodies to the respective antigen peptides on the membrane. The membrane was then washed with TBS containing 0.05% Tween 20 (TBST) for 5 minutes three times. The following procedure until color development was performed using ProtoBlot Western Blot AP System (Promega).

Anti-rabbit IgG antibody to which alkaline phosphatase was covalently bound (second antibody) was diluted 5000-fold with TBST. The membrane was immersed in this diluted solution at 4° C. for 2 hours to bind alkaline phosphatase to the antigen-first antibody binding product on the membrane through the second antibody. The membrane was washed with TBST for 5 minutes three times, then with TBS for 5 minutes twice. The membrane was immersed in a reaction mixture (100 mM Tris-HCl (pH 9.5), 100 mM NaCl, 5 mM MgCl₂) supplemented with 0.165 mg/ml of 5-bromo-4-chloro-3-indolyl phosphate (BCIP) and 0.33 mg/ml of nitro blue tetrazolium (NBT) and the existence of alkaline phosphatase on the membrane was detected by development of purple color. The reaction was terminated by immersing the membrane in water.

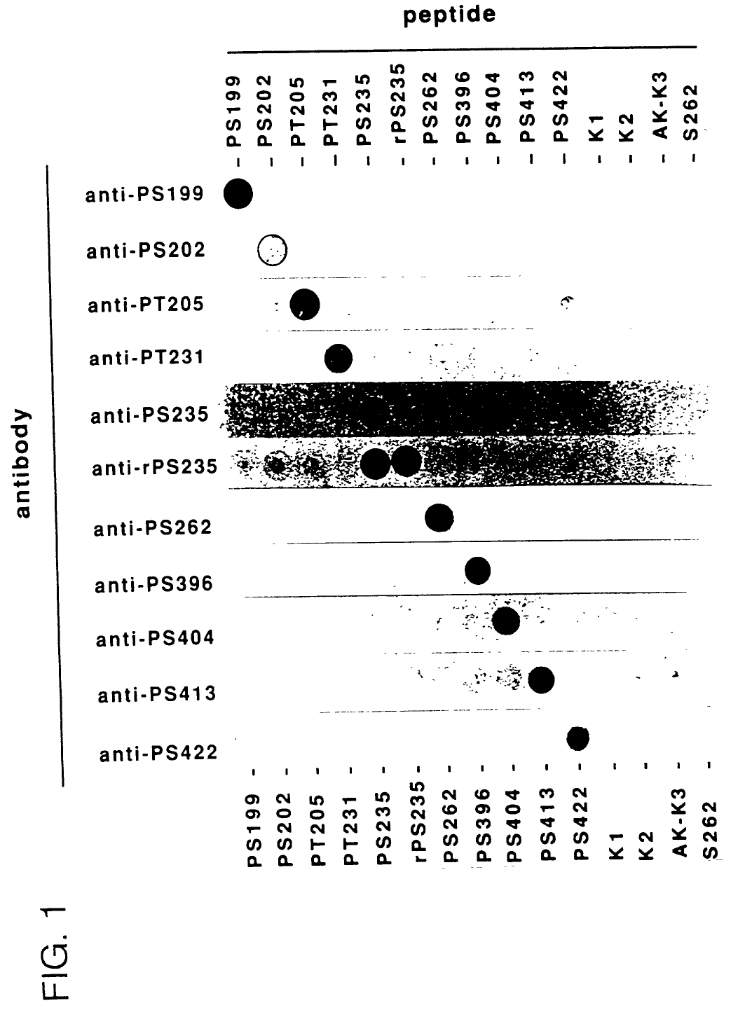

The first antibodies used in this Example were all rabbit antisera as they were. The same results were obtained using IgG obtained by purifying the antisera by affinity chromatography using a peptide column. The dilution of each antiserum was 1000-fold for anti-PS199, 250-fold for anti-PS202, 500-fold for anti-PT205, 250-fold for anti-PT231, 250-fold for anti-PS235, 25-fold for anti-rat PS235 (anti-rPS235), 500-fold for anti-PS262, 1000-fold for anti-PS396, 500-fold for anti-PS404, 500-fold for anti-PS413, and 500-fold for anti-PS422. The control peptides used were non-phosphorylated peptides including the amino acid sequence shown by SEQ ID NO: 19 (a peptide shown by K1, the 226th to the 240th amino acid residues, in SEQ ID NO: 1 in FIG. 1), the amino acid sequence shown by SEQ ID NO: 20 (a peptide shown by K2, the 191st to the 224th amino acid residues, in SEQ ID NO: 1 in FIG. 1), the amino acid sequence shown by SEQ ID NO: 21 (a peptide shown by AK-K3, the 384th to the 438th amino acid residues, in SEQ ID NO: 1 in FIG. 1), and the amino acid sequence shown by SEQ ID NO: 22 (a peptide shown by S262, the 257th to the 267th amino acid residues having a cysteine residue at the N-terminus, in SEQ ID NO: 1 in FIG. 1). These peptides were produced in the same manner in (a) and (b) in Production Example 1.

The results of dot blotting are shown in FIG. 1. The abscissa shows the peptide adsorbed by dotting it on the membrane and the ordinate shows the antibody. It shows that the respective antibodies obtained as described above are specifically bound to the corresponding phosphorylation sites.

### EXAMPLE 2

### Study on Reactivity of Antibody with Samples

(1) Preparation of Human Brain Extract

Human brain extracts were prepared from 8 cases of normal human brains and 19 cases of human brains of patients with Alzheimer's disease. The following procedure was all performed at 4° C.

1 g was sampled from chilled specimen of postmortem human cerebral cortex, cut into pieces with a razor in 3 ml of a TSinh solution [50 mM Tris-HCl (pH 7.6), 0.15 M NaCl, 0.5 mM DIFP (diisopropylfluorophosphate), 1 μg/ml antipain, 0.5 mM PMSF (phenylmethanesulfonyl fluoride), 1 mg/ml of TLCK (tricyl-lysine-chloromethyl ketone), 1 μg/ml of leupeptin, 0.1 g/ml of pepstatin], sonicated, and made into a suspension with a homogenizer. The resulting suspension was centrifuged at 80,000 rpm for 15 minutes to obtain a supernatant. Human IgG in this supernatant was removed with Protein G-Sepharose 4 Fast Flow (Pharmacia) and the resulting fraction was designated as TS fraction. The precipitate was sonicated in 2 ml of the above-described TSinh solution and made into a suspension with a homogenizer. The resulting suspension was washed by centrifugation at 80,000 rpm for 15 minutes and the thus-obtained precipitate was sonicated in 2 ml of TSinh containing 1% Triton X-100 and made into a suspension with a homogenizer. The homogenized product was centrifuged at 80,000 rpm for 15 minutes to obtain a supernatant (TX fraction). The precipitate was sonicated in 2 ml of TSinh containing 1% Triton X-100, made into a suspension with a homogenizer and washed by centrifugation at 80,000 rpm for 15 minutes. The resulting precipitate was sonicated in 2 ml of TSinh containing 2% SDS and homogenized to obtain a suspension. The suspension was centrifuged at 80,000 rpm for 15 minutes to obtain a supernatant (SDS supernatant fraction). The resulting precipitate was sonicated in 2 ml of TSinh containing 2% SDS, made into a suspension with a homogenizer, and washed by centrifugation at 80,000 for 15 minutes. The thus-obtained precipitate was sonicated in 2 ml of TSinh containing 2% SDS and made into a suspension (SDS precipitate fraction) with a homogenizer.

(2) Immunoblotting Using Phosphorylation Site-specific Antibodies

Laemmli's sample treatment solution (Nature 227, 680-685 (1970)) was added to each fraction obtained above. The mixture was heated at 95° C. for 5 minutes and subjected to SDS polyacrylamide gel electrophoresis. The resulting electrophoretic patterns were transferred to Immobilon P-membrane (Millipore). After blocking with TBS containing 5% skim milk for 2 hours, the membrane was reacted with the phosphorylation site-specific antibody obtained in Example 1 as a first antibody for 14 hours. The reaction mixture was washed with Tween 20 and TBS and treated with ProtoBlot AP (Promega) as a second antibody to develop color of the antibody-antigen reaction product on the membrane.

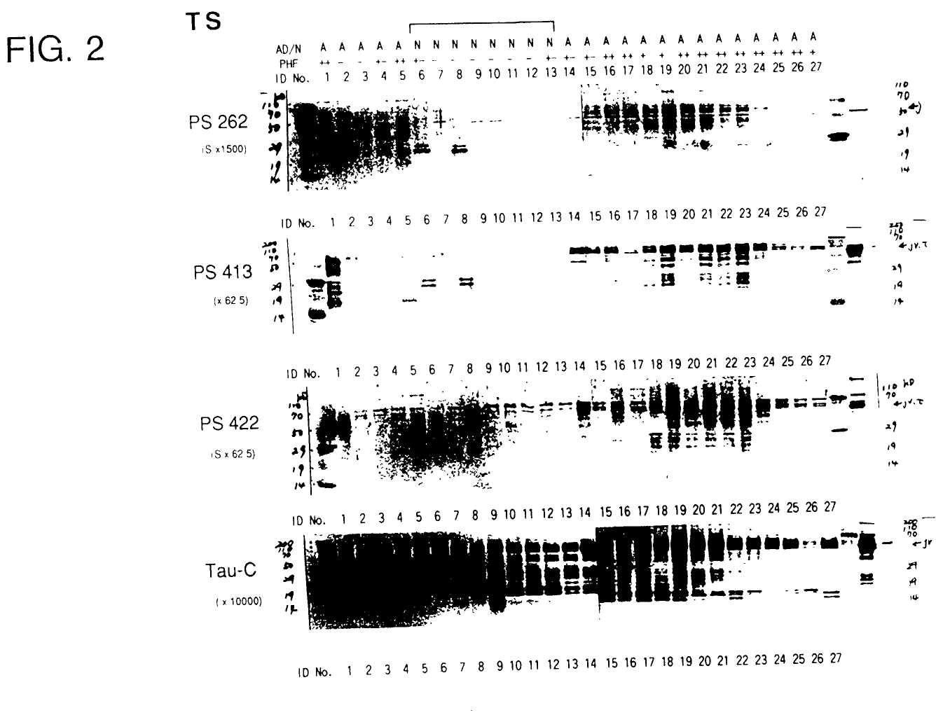

The first antibody was all rabbit IgG and dilution of the first antibody was shown by the following numerals after “x” under the names of the respective antibodies in FIGS. 2 to 4 with respect to Examples. Almost all of the first antibodies were previously purified by affinity chromatography using an antigen peptide column. However, PS262 and PS422 were used in the form of antisera and distinguished from others by indicating “S” under the name of antibodies in these figures.

Tau-N and Tau-C are peptides obtained in Production Examples 16 and 17 and correspond to the 2nd to the 12th and the 422nd to the 438th amino acid residues, respectively, of human tau protein represented by SEQ ID NO: 1 in the sequence listing, and used as controls showing the existence of tau protein.

A positive control used was whole brain extract of 8-day-old juvenile rat. Juvenile tau protein is known to be highly phosphorylated tau protein which is similar to that in the PHF (J. Biol. Chem. 268, 25712-25717 (1993)). Specifically, 0.75 g of 8-day-old juvenile rat brain was homogenized in 1.5 ml of medium containing 10 mM Tris-HCl (pH 7.4), 50 mM NaCl, 50 mM NaF, 1 mM EDTA, 1 mM EGTA, 50 mM β-glycerophosphoric acid, 0.1 mM Na₃VO₄, 1 mM PMSF, 1 μg/ml antipain, 1 μg/ml leupeptin, 0.1 μg/ml pepstatin, and 3 mM benzamidine. The homogenized product was centrifuged at 25,000 rpm for 20 minutes and a supernatant was recovered to serve as a positive control.

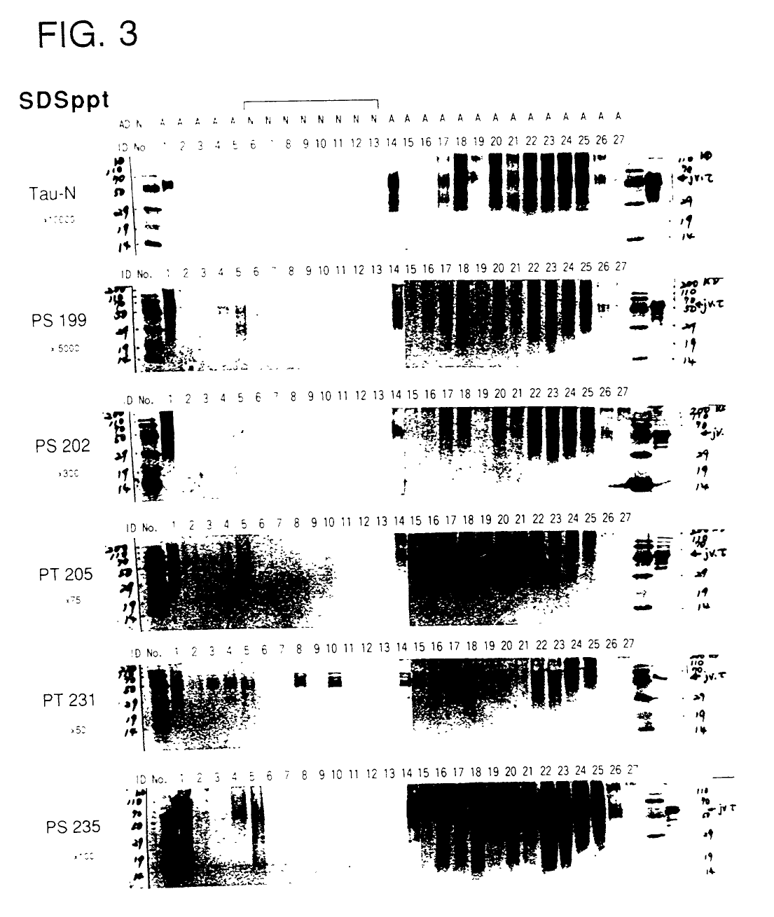

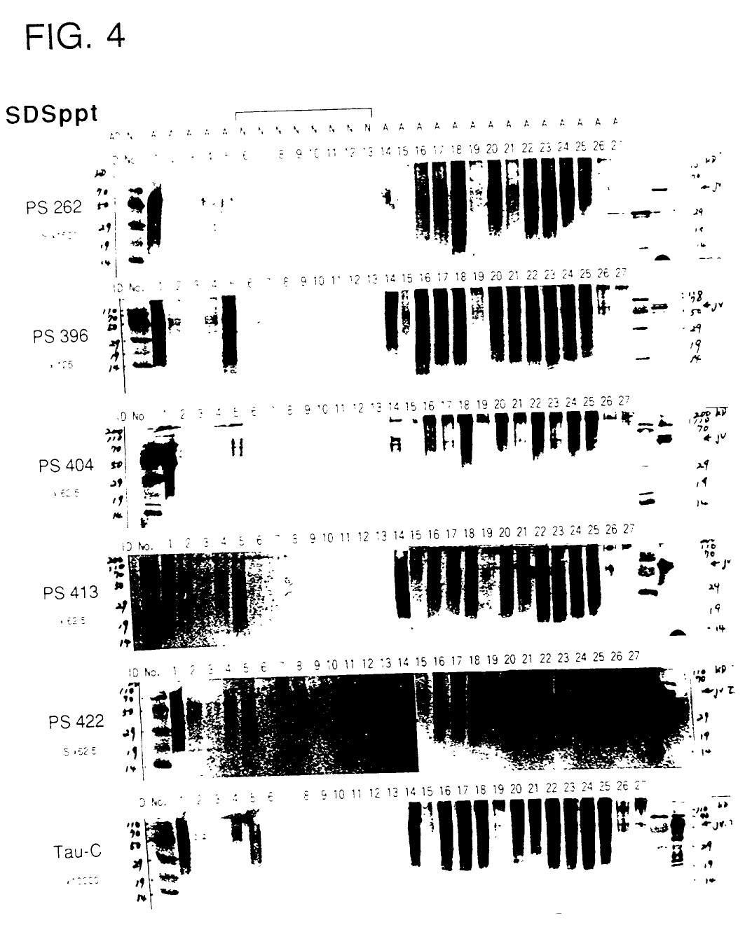

The results are shown in FIGS. 2, 3 and 4. FIG. 2 shows the result of immunoblotting of the TS fraction; FIGS. 3 and 4 show the result of immunoblotting of the SDS precipitate fraction. In these figures, “A” in lane AD/N stands for the result of brain extract from an Alzheimer's disease patient; “N”, for the result of normal brain extract; “M ”, for molecular weight markers; the next numerals for the molecular weight of the markers in terms of kD. Rat P8 lane stands for the immunoblotting result of the positive control and the band indicated by an arrow is phosphorylated tau protein band.

Any antibody did not react with the normal brain extract, but did react with the brain extract from an Alzheimer's disease patient. This indicates that the method of the present invention enables detecting Alzheimer's disease. The TS fraction contains easily soluble tau protein, whereas the SDS precipitation fraction contains less soluble tau protein. The method of the present invention enables detecting not only phosphorylated tau protein in tissues but also that in cerebrospinal fluid or blood thought to contain easily soluble tau protein. In other words, cerebrospinal fluid and blood can also be used as a sample as well as tissues.

### EXAMPLE 3

### Studies on Reactivity of Anti-human Phosphorylated Tau Protein Antibodies with a Sample by Radioimmunoassay (RIA)

(1) Preparation of ¹²⁵I-labeled Antigen

(a) Labeling by Bolton-Hunter Method

Peptides having a lysine residue at the amino terminus as shown in Production Examples 1 to 5 (SEQ ID NO: 3, 13, 15, 16, and 2 in the sequence listing) were labeled with ¹²⁵I in the following manner.

A solution of ¹²⁵I-Bolton-Hunter reagent in benzene (18.5 MBq, 500 μCi; Du Pont, NEX-120) was placed in a reaction test tube and benzene was vaporized in a stream of nitrogen. 20 μg of the peptide dissolved in 50 μl of 500 mM sodium chloride-160 mM borate buffer (pH 8.5) was added to the test tube and reacted for 2 hours under cooling with occasional stirring. 10 μl of 10% potassium iodide solution and 700 μl of 0.1% trifluoroacetic acid were added to the reaction mixture and the resulting mixture was purified by HPLC using a reverse phase column (internal diameter: 4 mm and length: 25 cm) packed with ODS (octadecylsilane)-bound silica. Elution was performed by a linear gradient of acetonitrile in 0.1% trifluoroacetic acid up to 60% at a rate of 1%/min. The flow rate of the eluate was 1 ml/min. The eluate was fractionated per 0.5 minute. A part of the fraction considered as a peak monitored by a radioactivity detector was measured with a scintillation counter to confirm a desired fraction. Phosphate buffer (Dulbecco's PBS(−), pH 7.3) containing 2% bovine serum albumin (BSA), 0.05% sodium azide, and 0.05% Tween 20 was added to the fraction. The resulting solution was divided into vials in small amounts containing 1,000,000 to 4,000,000 count/min (cpm) per vial and lyophilized.

(b) Labeling by Chrolamin-T Method

Peptide having a tyrosine residue at the amino terminus in place of a cysteine residue in the peptides as shown in Production Examples 6 to 14 (SEQ ID NO: 6, 11, 4, 12, 14, 7, 5, 8, and 9 in the sequence listing) were synthesized by the Fmoc method as used in Production Example 11 and the resulting peptides were labeled with ¹²⁵I in the following manner.

10 μg of the peptide was dissolved in 50 μl of 0.2 M phosphate buffer (pH 7.3),18.5 MBq of Na¹²⁵I (500 μCi; Du Pont, NEZ-033H) was added thereto, and the mixture was stirred. 3 μg of chrolamin-T (1 mg/ml in 0.2 M phosphate buffer; dissolved just prior to addition) was added thereto. The mixture was stirred and reacted at room temperature for 5 minutes. 17.5 μg of ascorbic acid (0.7 mg/ml 0.2 M phosphate buffer; dissolved just prior to addition) was then added thereto, the mixture was stirred, and the reaction was performed at room temperature for 1 minute. 10 μl of a 10% potassium iodide solution and 700 μl of 0.1% trifluoroacetic acid were added thereto. The resulting mixture was purified by reverse phase HPLC in the same manner as in (a) and fractionated. The desired fraction was divided into vials in small amounts and lyophilized.

(2) Competitive RIA

“Anti-PS262” among the antibodies prepared in Production Example 18 is exemplified below.

¹²⁵I-YPS-262 obtained in (1) was dissolved in RIA buffer (Dulbecco's PBS(−) containing 0.1% BSA, 0.05% sodium azide, and 0.05% Tween 20) and a 100 μl portion (about 10,000 cpm) was added to an assay tube. As a standard, 100 μl of RIA buffer containing peptide YPS262 whose amino acids were determined was also added within the range from 0 to 10000 fmol/ml. After 300 μl of the RIA buffer was added, 100 μl of “anti-PS262” diluted 10,000-fold with the RIA buffer was further added and the mixture was stirred. The mixture was incubated overnight at 4° C. 100 μl of normal rabbit serum diluted 128-fold with the RIA buffer, 100 μl of anti-rabbit IgG goat serum diluted 32-fold, 200 μl of Dulbecco's PBS(−) containing 8% polyethylene glycol (PEG6000) and 0.2% cellulose powder (Avicel, Registered Trademark) were added thereto. The mixture was stirred and incubated at 4° C. for 30 minutes then centrifuged at 4° C. at 3000 rpm for 20 minutes. The resulting supernatant was removed by suction and the radioactivity of the precipitation was measured with a scintillation counter.

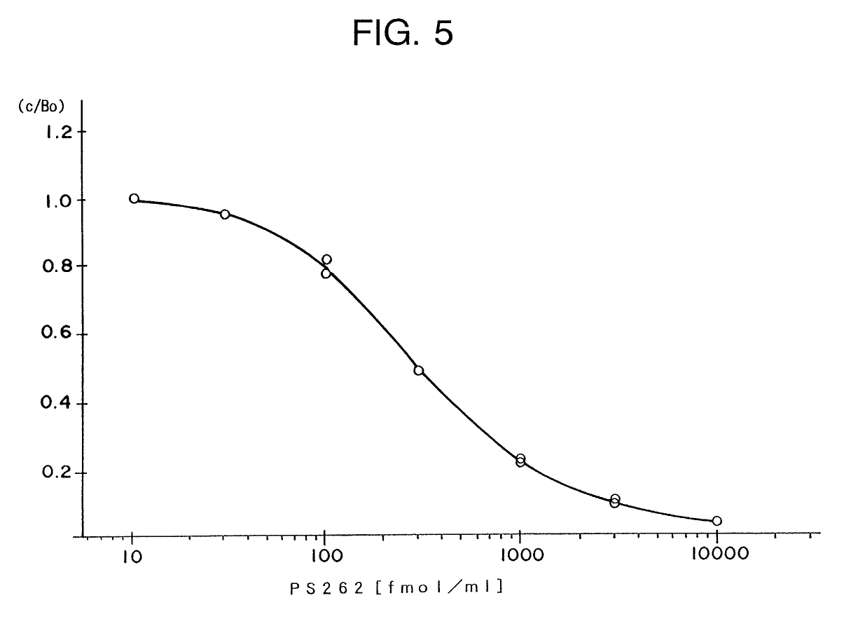

A calibration curve was prepared by plotting the concentration of the standard substance (fmol/ml) as abscissa and the ratio of the count at each concentration of the standard substance to the count at 0 fmol/ml of its concentration as ordinate. FIG. 5 shows this calibration curve. FIG. 5 indicates that the peptide can be measured 30 fmol/ml at minimum with good accuracy by the method of the present invention.

Furthermore, a derivative S262 of the peptide shown by SEQ ID NO: 10 in the sequence listing, in which cysteine is not added and serine is not phosphorylated, was synthesized by the Fmoc method in the same manner as in Production Example 11 and was used to evaluate the specificity of the antibody anti-PS262. The count did not decrease even when S262 was added to 2000 fmol/ml, indicating that the antibody of the present invention specifically recognized PS262 containing phosphorylated serine.

(3) Measuring Phosphorylated Tau Protein in Cerebrospinal Fluids from Patients

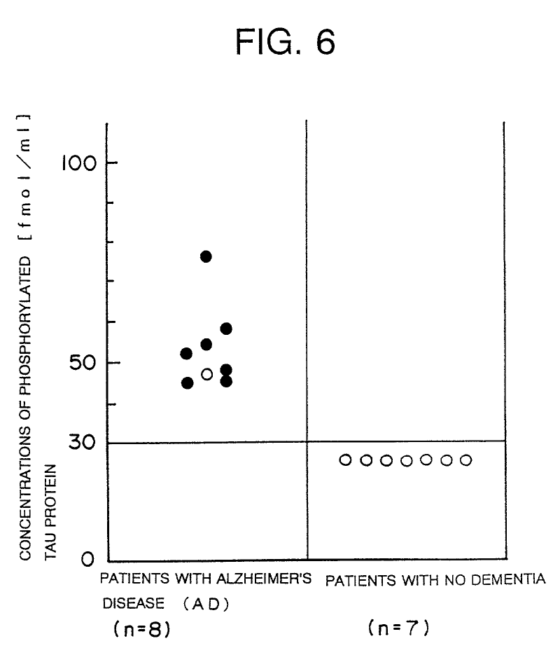

The concentrations of phosphorylated tau protein in cerebrospinal fluids from 8 patients with Alzheimer's disease (AD) were measured using the method and the calibration curve in (2). The concentrations of phosphorylated tau protein in cerebrospinal fluids from 7 non-dementia patients were also measured as a control (CTL). The results are shown in FIG. 6. As shown in FIG. 6, phosphorylated tau protein was not detected in any of CTL, while 45 to 75 fmol/ml of phosphorylated tau protein was detected in AD.

### Industrial Applicability

The present invention provides antibodies that specifically recognize phosphorylated tau protein using as an immunogen a partial peptide containing a phosphorylation site of phosphorylated tau protein in the PHF. Phosphorylated tau protein in brain extracts and tissue sections can be detected using the antibody obtained as above. Furthermore, the method of measuring phosphorylated tau protein using this antibody of the present invention enables simply measuring phosphorylated tau protein in body fluids and is useful for detecting Alzheimer's disease.

What is claimed is:

1. Antibodies obtained by using as an immunogen a partial peptide comprising phosphorylation sites of phosphorylated tau protein in a paired helical filament.

2. Antibodies defined in claim 1, recognizing the phosphorylation site(s) of phosphorylated tau protein which site(s) are one or more amino acid residues selected from serine at position 198, serine at position 199, serine at position 202, threonine at position 205, threonine at position 231, serine at position 235, serine at position 262, serine at position 396, threonine at position 403, serine at position 404, serine at position 409, serine at position 412, serine at position 413, and serine at position 422 of an amino acid sequence of SEQ ID NO: 1.

3. Antibodies defined in claim 1, recognizing the phosphorylation site(s) of phosphorylated tau protein which site(s) are one or more amino acid residues selected from serine at position 199, serine at position 202, threonine at position 205, threonine at position 231, serine at position 235, serine at position 262, serine at position 396, serine at position 404, serine at position 412, serine at position 413, and serine at position 422 of an amino acid sequence of SEQ ID NO: 1.

4. Antibodies defined in any one of claims 1 to 3, which recognize the partial peptide comprising the phosphorylation site(s) which peptide comprises an amino acid residue at the phosphorylation site(s) and plural amino acid residues before and/or after the phosphorylation site.

5. Antibodies defined in any one of claims 1 to 4, which recognize the partial peptide comprising the phosphorylation site, which peptide has an amino acid sequence of any one of SEQ ID NO: 2 to SEQ ID NO: 16.

6. A reagent kit used for detecting Alzheimer's disease, comprising at least the antibodies as defined in any one of claims 1 to 5.

7. Methods for detecting Alzheimer's disease, which are characterized by examining reactivity of the antibodies, as defined in any one of claims 1 to 5, with a sample from an individual suspected of Alzheimer's disease.
